(19) United States
(12) Patent Application Publication
(10) Pub. No.: US 2025/0303093 A1
(43) Pub. Date: Oct. 2, 2025

(54) APPARATUS AND METHOD FOR TREATMENT OF SLEEP DISORDER BREATHING

(71) Applicant: Myosa Pty Ltd, Helensvale QLD (AU)

(72) Inventor: Christopher John Farrell, Helensvale QLD (AU)

(21) Appl. No.: 19/238,948

(22) Filed: Jun. 16, 2025

Related U.S. Application Data
(63) Continuation-in-part of application No. 17/259,531, filed on Jan. 11, 2021, now Pat. No. 12,364,829.

(30) Foreign Application Priority Data
Jul. 20, 2018 (AU) 2018902632

(51) Int. Cl. A61M 16/06; A61F 5/56; A61M 16/08

(52) U.S. Cl. CPC A61M 16/06; A61F 5/566; A61M 16/0816

(57) ABSTRACT

An adapter for connecting an intra-oral appliance on a patient to a CPAP machine is disclosed. The adapter includes an air inlet portion forming an air inlet and an air outlet portion forming an air outlet, and an air passage extending from the air inlet to the air outlet. The air outlet portion may have a complementary configuration to an air inlet member of the intra-oral appliance and be sized to be received snugly within the air inlet member when in use. The air inlet portion may be operatively connected to an outlet of an air tube for connection to the CPAP machine. An apparatus for carrying out CPAP treatment including an intra-oral appliance and an adapter operatively coupled to the oral appliance is also disclosed.

### CROSS REFERENCE TO RELATED APPLICATIONS

This application is a Continuation-in-part of U.S. patent application Ser. No. 17/259,531 filed Jan. 11, 2021, which is a U.S. National Stage Entry of International Patent Application No. PCT/AU2019/050757 filed Jul. 19, 2019, which claims priority to Australian Patent Application No. 2018902632 filed Jul. 20, 2018, all of the aforementioned applications are hereby incorporated by reference in their entireties.

### FIELD

The present disclosure relates broadly to an apparatus and method for use in the treatment of sleep disorder breathing (SDB).

This invention relates to an adapter for connecting an intra-oral appliance to an air hose leading to an air supply machine. The invention also extends to an apparatus including the adapter for treating a patient, and a method of treating a patient with the adapter operatively coupled to an intra-oral appliance.

This invention relates particularly, but not exclusively to an adapter for connecting an intra-oral appliance to an air hose leading to a CPAP machine for delivering continuous positive air pressure. It also extends to an apparatus including an adapter for coupling to a CPAP machine and carrying out CPAP treatment. It will therefore be convenient to hereinafter describe the invention with reference to this example application. However, it is to be clearly understood that the invention is not to be limited to CPAP treatment. It extends to intra-oral appliances and adapters used for all other purposes.

### BACKGROUND

Over the past two decades, the medical and dental profession has become more aware of breathing disorders and in particular sleep disorder breathing as a major contributor to several health problems. Previously, it was considered that snoring was merely a sleeping habit, but it is now known that this can lead to more severe disorders like Obstructive Sleep Apnea (OSA). OSA has been associated with heart disease, strokes and chronic daytime tiredness and spontaneous sleeping. OSA and snoring fall under the generic term of Sleep Disorder Breathing (SDB).

Many clinicians regard SDB as a wide spectrum of sleep-related breathing abnormalities related to an increase in upper airway resistance that includes snoring, upper airway resistance syndrome (UARS), and obstructive sleep apnea-hypopnea (OSA). This philosophy suggests that a person who snores may be exhibiting the first manifestation of SDB and that snoring should not be viewed as normal. This is supported by experimental studies showing increasing airway collapsibility during sleep with a progression from normal sleep, to snoring, to UARS, and then finally to OSA. Snoring is one of the most common forms of SDB. After sleep apnea syndrome was recognized, snoring began to be viewed as an important clinical symptom. Although it is by far the most common symptom of sleep apnea, not all patients who snore have sleep apnea.

The pathogenesis of OSA involves a combination of reduced upper airway size and altered upper airway muscle activity, which causes oral tissue to collapse and an obstruction to occur in an associated airway. When a person is awake, their muscles hold the pharyngeal airway open, but these muscles can relax when they are asleep causing the airway to collapse. Other factors which are thought to contribute to OSA include a patient's weight particularly when they are obese, tongue size, soft palate volume, a retrognathic mandible, and an anteroposterior discrepancy between the maxilla and the mandible.

Both snoring and OSA are generally caused by a blockage of the pharyngeal airway. This can occur due to excess tissue when various muscles of the body, including the tongue, relax. As the tongue relaxes, it moves posteriorly, at least partially blocking the pharyngeal airway. When the pharyngeal airway is blocked, air is forced through the airway with increased velocity on exhalation thereby causing a vibration of the tongue, tissue, or other obstruction, thereby creating noise.

Snoring is caused by the partial obstruction of breathing during sleep while OSA occurs when the tongue and soft palate collapse onto the back of the throat and completely block the pharyngeal airway, thereby interrupting breathing and restricting flow of essential oxygen. Snoring is common in people who breathe through their mouth when asleep. Mouth breathing causes the mandible to drop and decreases the area of the pharyngeal airway. It also causes the tongue to be pushed back into the throat, thereby creating the obstruction associated with SDB.

A typical medical treatment for OSA has been the delivery of Continuous Positive Air Pressure Appliance (CPAP) from a CPAP machine. The CPAP treatment uses a positive air pressure to blow air through the nose and into the upper airways to prevent upper airway collapse during sleep. The positive air pressure is generated by a pump and is applied through a small nasal mask which fits over the nose, nasal pillows, or a face mask that fits over the nose and mouth.

When pressure is applied to the nose, the uvula and soft palate partially block off the mouth. While some air may escape through the mouth, if the mouth is kept closed a seal can be obtained. Air leakage through the mouth is known as “mouth leak” and can lead to a large unidirectional flow of air in through the nose and out through the mouth. The driving force for the mouth leak air flow is the positive air pressure of the CPAP. Full face (oronasal) masks may be used to resist mouth leak. Alternatively, chinstraps may be used to hold the mouth closed. However, neither solution is conducive to a comfortable night's sleep, nor can it be considered a satisfactory solution.

It is important that a CPAP mask provides a good seal against the face and is kept firmly in place by head straps. However, the tighter that a full face mask is fitted, the more the mandible is forced back into the airway as well as compromising the tongue position. This makes the SDB and OSA worse. The problem is well known with the treatment of OSA.

Further in practice, the masks are uncomfortable, and some patients feel claustrophobic while wearing face masks. For this reason, nasal only masks are preferred, but these masks can suffer from either mouth leak or inefficiency if the patient has a nasal obstruction.

Many patients cannot tolerate CPAP when their nasal breathing is obstructed. This may be due to a number of factors including nasal or sinus structure abnormalities like a deviated septum, swollen turbinates and problems with the upper palate and nasal congestion.

CPAP can have undesirable side effects that also lead to non-compliance or intolerance. Such side effects include nasal irritation that can cause congestion and mucosal dryness. This can lead to patients unconsciously taking off the mask prematurely. Nasal irritation is exacerbated by mouth leak. Poor adherence or compliance (30-60%) to CPAP is a recognized limiting factor in treating OSA, leaving patients at risk for co-morbid conditions and impaired quality of life. Compliance to CPAP therapy is defined as CPAP for at least 4 hours for 70% of nights. This definition alone has an inherent non-compliance factor built in, as it would be optimal for the CPAP treatment to be used for the entire sleeping period to limit apnea and hypopnea events. In conclusion, the CPAP treatment that is now widely used has many inherent disadvantages, but it is accepted that this is the best treatment that can be provided to a SDB patient.

Alternatives to CPAP treatment for patients who are non-compliant include the use of intra-oral appliances or Dental Sleep Appliances (DSA). DSAs for alleviation of SDB symptoms are considered less effective than CPAP and not suitable for more severe cases. However, they are more convenient, easier to use and certainly more portable. The compliance factor has brought the attention of the medical profession to view DSA's as the primary treatment for SDB for moderate to more severe cases who have a compliance issue with the CPAP regime.

There are many types of DSA's with varying designs and the most common is Mandibular Advancement Device (MAD). The principle behind a MAD device is that advancing the mandible in an anterior position relative to the maxilla during sleep opens the pharyngeal airway by indirectly urging the tongue forward to stimulate activity of the muscles in the tongue and thereby also increases the forward rigidity of the tongue. Since the tongue attaches to the posterior portion of the mandibular symphysis, advancing the mandible forward relative to the maxilla also pulls the tongue forward, preventing the tongue from obstructing the pharyngeal airway. MAD devices therefore function to move the mandible, and hence the tongue forward to open up the oropharynx. Snoring is believed to decrease proportionally with the increase in airway size or diameter.

Some MAD devices comprise two hinged parts that are adjustably connectable to allow for titration of the amount of advancement while others are formed from a single piece of thermoplastic forming a living hinge. A recognized advantage of hinged devices is that they allow the mouth to open for unrestricted breathing. It is considered very important that breathing is not restricted for mouth breathers as the object of the prior art MAD devices is to increase the amount of airflow.

Other devices are known as tongue retaining devices that work by pulling the tongue forward so as to open the airway, with little or no mandibular advancement when compared with the MAD devices. It will be appreciated that by pulling the tongue forward, it is also not possible to breathe through one's mouth and/or many devices block the mouth completely. Such devices are uncomfortable and have poor compliance rates. And are completely unsuitable for patients with nasal obstruction, congestion or irritation. Further these MAD devices pose potentially damaging effects. Most single piece devices fit over both the maxillary and mandibular teeth and are typically held nearly stationary, thereby restricting movement, causing discomfort, and potentially causing permanent repositioning of the jaw.

The temporomandibular joint (TMJ) connects the mandible to the skull. The mandibular condyle is received within the superior synovial cavity. The TMJ is flexible, allowing the mandible to move smoothly up and down and side to side and enabling a person to talk, chew and yawn. Muscles attached to and surrounding the TMJ control the position and movement of the mandible. The ongoing use of devices that restrict the natural lateral movements of the mandible, as well as anterior and posterior movement of the mandible, can potentially aggravate the TMJ and related facial musculature. Still further, advancing the mandible will place stress on the TMJ as the mandibular condyle is anteriorly displaced relative to its normal position. This can lead to TMJ disorder which covers a group of conditions that cause pain and dysfunction in the jaw joint and muscles that control jaw movement.

There are therefore serious concerns amongst the dental community about the medium to long term effects of MAD devices that advance the mandible too far. These effects can include adverse changes in dental occlusion, damage to teeth and potential damage to the TMJ. However, it is believed that the high priority accorded to correcting snoring and SDB by medical practitioners trumps this side effect that needs to be accepted for the overall benefit conferred by this treatment.

A healthy TMJ is a prerequisite for MAD use and patients with existing TMJ disorders are generally cautioned against using a MAD appliance, as it can exacerbate their TMJ condition. MAD appliances are based on the same orthodontic appliance principles which are designed to correct a class II malocclusion. In a class II malocclusion, there is a misalignment of the teeth on the mandibular and maxillary dental arches with the upper teeth being forward of the lower teeth in what is commonly known as an overbite. Orthodontic appliances for the treatment of class II malocclusions in pre-adolescent patients advance the mandible and stimulate and enhance growth and development of the mandible. These appliances also place an equal and opposite retractive force on the maxilla and this restrains development of the maxilla.

It is generally believed that the greater the level of mandibular advancement achieved by a MAD device, the more effective is the treatment for SDB. Advances of between 50% and 75% of maximum mandibular protrusions are recommended and physiological protrusion lengths between about 7 mm and 12 mm are typical. The American Academy of Dental Sleep Medicine (AADSM) has published a report on what features define an effective oral appliance for the treatment of OSA. One of the features defined is that the appliance should permit protrusive advancement over a range of at least 5 mm. It is considered desirable to be able to gradually advance the mandible over time with treatment to reduce pain and soreness and alleviate TMJ pain.

These recommendations have been based upon several factors and in particular pulse oximetry which measures hemoglobin O₂ saturation. This measurement is based upon the generally held belief in the MAD device art that physically opening the airways and allowing more air to be inhaled is sufficient to treat SDB. It may be appreciated that using an oral appliance designed for treating a class II malocclusion by advancing the mandible and retracting the maxilla would have the same effect on a person with normal occlusion.

The maxilla determines an effective horizontal dimension of the pharynx and in particular the upper pharynx. Maxillary constriction has been reported to be associated with narrowing of the pharyngeal airway and may play a role in the etiology of OSA. Surgical maxillary expansion may be an effective procedure for widening nasal cavities and decreasing nasal airway resistance which can lead to improvement in nasal breathing.

More recently, a modification to a conventional MAD device or appliance was described in Australia Patents Numbers AU2012255625 and AU2015240431. The disclosed appliance has a rigid U-shaped body and an air inlet/outlet opening extending from the front of the U to allow for mouth breathing. The appliance has enclosed channels extending from the inlet at the front along the full length of the arms so each that extends from the front delivers inhaled air from the inlet to the posterior of the oral cavity in the vicinity of a junction between the hard and soft palates. In this way, the device bypasses any obstruction of the airway by the tongue or other soft tissue. The MAD device or appliance has a lingual flange for engaging mandibular teeth for mandibular advancement. The appliance is considered to be particularly suitable for patients with nasal congestion or obstruction who are intolerant of CPAP.

WO2015/149127 describes a similar device in which the lingual flange is moveable by a screw device such that the degree of mandibular displacement can be titrated.

WO2017/020079 also describes a similar device to that above and further includes a removable tongue retainer for holding the tongue forward between the teeth.

WO2017/165918 further describes a device having air channels for delivering air to the posterior of the oral cavity. In this disclosure, the body is made in two parts connected via an adjustment mechanism so as to adjust the degree of mandibular displacement.

Each of the appliances or devices described in the above patent documents must be custom made specifically for each individual. A method involving 3D scanning of the mouth of a user is described. The scanned information is then used to manufacture a customized device by additive manufacturing such as 3D printing of a metallic material such as titanium or stainless steel. Electron beam melting (EBM) is a technique that is specifically mentioned which uses electron beams to build up metal layers one at a time. In the appliance the metal bodies are encased in a soft plastics material. It is important that the enclosed channels that pass through the arms of the appliance can deliver a sufficient and uninterrupted flow of air to the posterior of the oral cavity. The rigid titanium body ensures that this occurs. The rigidity of the body also means that accurate scanning of a user's mouth must be made to ensure an accurate fit with comfort and compliance. It will be also appreciated that 3D scanning and custom manufacturing of the appliances by 3D printing of titanium as described above is time consuming and expensive. It follows that appliances manufactured thereby may be unavailable to many SDB patients.

Conventional non-customized DSAs may be injection molded from a flexible thermoplastics material such as ethylene vinyl acetate (EVA) that softens below 100° and are therefore self moldable to a user's dentition. When molding, a user presses the teeth on the softened EVA. If air channels are contained in the arms of the body between the teeth there is a risk of distortion of the channels that may compromise uniformity of air flow.

### SUMMARY

Applicant recognizes that it would be beneficial to provide an intra-oral appliance and an efficient apparatus for coupling the intra-oral appliance to a CPAP machine for delivering positive air pressure into the mouth of a patient with an intra-oral appliance.

According to one aspect of the invention there is provided an adapter for connecting an intra-oral appliance on a patient to a CPAP machine, the adapter comprising:
an air inlet portion forming an air inlet and an air outlet portion forming an air outlet, and an air passage extending from the air inlet to the air outlet,
wherein the air outlet portion has a complementary configuration to an air inlet member of the intra-oral appliance and is sized to be received within the air inlet member in use; and
the air inlet portion is operatively connected to an outlet of an air tube for connection to the CPAP machine.

The air outlet portion may comprise a curved upper major surface and a curved lower major surface, and the upper and lower major surfaces may be joined along their side edges.

The air outlet portion may have a flattened configuration, e.g. a substantially elliptical configuration, broadly corresponding to the shape of the patient's mouth.

The air outlet portion may comprise an engagement formation or catch formation for releasably engaging the air inlet member of the intra-oral appliance and holding the adapter to the intra-oral appliance.

The air outlet portion may comprise a curved upper major surface and a curved lower major surface, and the upper and lower major surfaces may be joined along their side edges.

The air outlet portion may have a flattened configuration, e.g. a substantially elliptical configuration, broadly corresponding to the shape of the patient's mouth.

The air outlet portion may comprise an engagement formation or catch formation for releasably engaging the air inlet member of the intra-oral appliance and holding the adapter to the intra-oral appliance.

The catch formation may be positioned at a free or terminal end of the air outlet portion.

The catch formation may be configured to sit behind an inner end of the air inlet member of the intra-oral appliance in use when the appliance is engaged with the adapter.

The air outlet portion may further comprise an internal passage support, e.g. in the air passage, for holding the air passage open during use of the adapter.

The internal passage support may comprise a support post located in the air passage and extending across the air passage adjacent to the air outlet.

The adapter may further comprise an adapter wall or adapter flange extending transversely outward beyond the periphery of the air inlet portion and air outlet portion. The adapter wall may be axially positioned between the air inlet and air outlet portions.

The adapter wall may have a curved configuration for complementing the curvature or contour of a patient's mouth and face, around the mouth of the patient.

The adapter wall may have a substantially elliptical or ellipsoid configuration, and the cross-sectional shape of the adapter wall may be elongated in the direction of the upper and lower major surfaces of the air outlet portion.

The adapter may include any one or more features of the adapter described or defined in any other aspect of the invention.

According to another aspect of the invention there is provided an apparatus for carrying out CPAP treatment, comprising:
intra-oral appliance comprising an appliance body forming an upper arch receiving channel, and an air inlet member forming an air inlet extending forward from the appliance body, an air outlet member forming an air outlet opening configured to locate above the patient's tongue in use, and an air flow passageway extending between the air inlet member and the air outlet member for placing the air inlet opening and air outlet in fluid communication; and
an adapter mounted on the intra-oral appliance, the adapter comprising an air inlet portion forming an air inlet and an air outlet portion forming an air outlet, and an air passage extending from the air inlet to the air outlet, wherein the air outlet portion has a complementary configuration to the air inlet member of the intra-oral appliance and is received therein, and the air inlet portion is operatively connected to an outlet of an air tube for connection to the CPAP machine.

The air outlet portion of the adapter may be substantially rigid.

The air outlet portion may have a substantially elliptical cross-sectional configuration that is configured to be snugly or tightly received within the air inlet member of the intra-oral appliance.

The air outlet portion of the adapter may comprise a substantially constant cross-sectional configuration along its length.

The adapter may further comprise an internal passage support for holding the air passage in the outlet portion open which is received in the air inlet member during use.

The air outlet portion may comprise an engagement formation releasably engaging the air inlet member of the intra-oral appliance, and the engaging formation may be positioned at a terminal end or free end of the air outlet portion, and behind an inner end of the air inlet member.

The air inlet member may be formed of a resilient and deformable material that can be deformed in use. The air inlet member may have a substantially constant elliptical cross-sectional configuration along its length that is sized and configured to enable the patient's lips to form a seal about the air inlet member.

Thus, the oral appliance is held in place by the teeth within the arch receiving channels and the seal is provided by the lips about the air inlet member that forms part of the oral appliance that receives the air outlet portion of the adapter.

The adapter may comprise an adapter wall extending transversely outward beyond a periphery of the air inlet and air outlet portions, and the adapter wall may be axially positioned between the air inlet and air outlet portions.

The adapter wall may comprise an elliptical configuration for corresponding to the shape of the mouth of a patient, and the adapter wall may have a curvature across its surface that complements a curvature or contour on the surface of the patient's face around their mouth.

The outer wall of the appliance body and the adapter wall may be configured to be positioned on respectively inner and outer sides of the patient's lips in use and help to resist the leakage of air out through a mouth opening of the patient, e.g. during the application of positive air pressure.

The outer wall and the adapter wall may each cover at least part of the patient's mouth and thereby help to resist the leakage of air out of the mouth opening when air is passed through the intra-oral appliance and into the mouth of the patient during treatment by the application of positive air pressure.

The air outlet member may be configured to abut or be close to the patient's hard palate, and the air outlet member may be formed of a resilient material that can be deformed in use into contact with the patient's hard palate.

The air outlet of the air outlet member may have a substantially elliptical configuration. Further, the air outlet member may taper outwardly along its length in a direction towards the air outlet opening.

In addition to the air inlet member and the air outlet member, the appliance body may also be formed of a resilient material that is capable of deformation in use

Conveniently, the entire oral appliance may be integrally formed as a unitary article made of a resilient material that is capable of deformation in use. Optionally, the resilient material may comprise a medical grade silicone material.

The air flow passageway of the intra-oral appliance may comprise an intermediate part, e.g. intermediate the air inlet and air outlet members, extending upward and posteriorly from the air inlet member to the air outlet member to accommodate the patient's tongue.

The intermediate part may curve upward and posteriorly from the air inlet member to the air outlet member to accommodate the tongue.

In addition to the upper dental arch receiving channel for receiving the maxillary arch, the appliance body may further comprise a lower dental arch receiving channel for receiving the mandibular arch of the patient.

Further, the lower dental arch receiving channel may be advanced slightly relative to the upper dental arch receiving channel relative to a position representing correct dental occlusion. This helps to advance the mandible when the intra oral appliance is fitted to a patient.

The apparatus may further include an air tube operatively connected to the air inlet portion of the adapter. The air tube may comprise a flexible air hose, e.g. a lightweight air hose made of polyethylene.

The apparatus may further include a CPAP machine operatively coupled to the air tube.

The adapter may include any one or more features of the adapter described or defined in any other aspect of the invention.

The intra-oral appliance may include any one or more features of the adapter described or defined in any other aspect of the invention.

According to another aspect of the invention there is provided a method of treating a patient with CPAP comprising fitting an intra-oral appliance and connected adapter as defined in in another aspect of the invention, the method comprising:
coupling the adapter to a CPAP machine; and
delivering positive air pressure from the CPAP machine to the patient through the adapter and intra-oral appliance into the upper airways of the patient to resist the obstruction of the upper airways of the patient.

The method may be implemented on a patient at nighttime while the patient is sleeping. Further, the method may be repeated daily at nighttime.

The intra-oral appliance may include any one or more features of the intra-oral appliance described or defined in any other aspect of the invention.

The adapter may include any one or more features of the adapter described or defined in any other aspect of the invention.

According to yet another aspect of the invention there is provided an oral appliance for treating sleep disorder breathing in a patient, the oral appliance comprising:
a U-shaped appliance body with a front section and two arms, the U-shaped appliance body including an inner wall and an outer wall, a web interconnecting the inner wall and the outer wall to define an upper dental arch receiving channel;
an air inlet member extending forward of the front section of the U-shaped appliance body, the air inlet member defining an air inlet opening that is spaced from the U-shaped appliance body for intake of air during inhalation;
an air outlet member extending from an upper portion of the inner wall that is located in the patient's mouth above the patient's tongue when in use, the air outlet member being configured to abut or be close to the patient's hard palate, the air outlet member comprising an air outlet, configured for delivering inhaled air between the patient's tongue and hard palate, and the delivered air is directed towards the patient's posterior oral cavity; and
an air flow passageway for communication of the air inlet opening with the air outlet.

The oral appliance may comprise a curved intermediate part between the air inlet member and the air outlet member for communicating the air inlet opening with the air outlet member.

The oral appliance may comprise a curved intermediate part between the air inlet member and the air outlet member for communicating the air inlet opening with the air outlet member.

The curved intermediate part may curve up from the air inlet member to the air outlet member to provide space for the tongue. Further, the curved intermediate part may have a smaller cross-sectional area for air flow therethrough than the cross-sectional area for air flow of the air inlet member.

The air outlet member may have a curved upper wall and a curved lower wall, and the upper and lower walls may form an air outlet having an elliptical configuration.

The curved upper and lower walls of the air outlet member may diverge in a direction towards the air outlet, and the air outlet member may have side edges that diverge in a direction towards the air outlet. The air outlet member may be angled up at an angle of 30 to 45 degrees to an axis passing through the web of the U-shaped body.

The air inlet opening may be sized to have a cross-sectional area for breathing that allows the patient to breathe fully through their mouth.

The air inlet member may be configured to allow the patient's lips to form a lip seal with the air inlet member about the circumference of the air inlet member.

The air inlet member may have an elliptical cross-sectional configuration that is constant and does not change along its length. Instead, the air inlet member may have an elliptical frusto-conical configuration comprising side walls that taper from the air inlet opening towards the appliance body.

The air flow passageway may comprise a constricted part between the air inlet member and the air outlet member.

The cross-sectional flow area of the air inlet member between the air inlet and the constricted part may be between about 200 mm² and about 400 mm², and the cross-sectional flow area of the constricted part may be between about 50 mm² and about 100 mm².

The air outlet member may have a degree of resilience that enables it to be deformed to contact or press against the patient's hard palate and direct inhaled air flow above the patient's tongue.

The appliance body may further comprise a lower dental arch receiving channel, and the upper and lower dental arch receiving channels may be configured so that when the oral appliance is fitted in the patient's mouth, the patient's mandible is advanced.

The patient's mandible may be advanced a distance of about 5 mm, or less. In at least one embodiment the patient's mandible may be advanced about 1 mm to about 3 mm.

According to yet aspect of the invention there is provided a combination comprising an oral appliance as defined in any other aspect of the invention, and an adapter comprising an adapter inlet and an adapter outlet, and an adapter air passage between the adapter inlet and the adapter outlet, wherein the adapter outlet is configured for operative coupling to the air inlet opening of the appliance, and the adapter inlet is configured for operative coupling to an external air supply, whereby to place the external air supply in fluid communication with the air flow passageway of the appliance.

The oral appliance may include any other features, or combination of features, of the oral appliance in any other aspect of the invention.

According to another aspect of the invention, there is provided a method of treating symptoms of sleep disorder breathing (SDB) in a patient comprising: providing an oral appliance as disclosed in any one of the preceding aspects of the invention and causing the patient to wear the oral appliance whilst sleeping.

The SBD that is treated may be snoring or obstructive sleep apnea (OSA). In this method, the appliance may be worn for at least three hours at nighttime while the patient is sleeping.

The oral appliance may include any other features, or combination of features, of the appliance in any other aspect of the invention.

According to yet another aspect of the invention there is provided a method of treating symptoms of sleep disorder breathing (SDB) in a patient comprising:
providing a combination of an oral appliance and an adapter as defined in any other aspect of the invention, and connecting the adapter inlet to a CPAP machine; and
fitting the oral appliance to the patient, and causing air from to flow from the CPAP machine through the adapter air passage and the air flow passageway in the oral appliance, into the patient's mouth,
wherein the air flowing into the patient's mouth assists in opening the patient's airways while they are sleeping.

The oral appliance may include any other features, or combination of features, of the appliance in any other aspect of the invention.

The present disclosure also extends to an oral appliance for the treatment of sleep disorder breathing in a patient, the oral appliance comprising:
a U-shaped appliance body with a front section and two arms, the appliance body including an inner wall and an outer wall, a web interconnecting the inner wall and the outer wall so as to define an upper dental arch receiving channel;
an air inlet opening on the front section of the appliance body for intake of air during inhalation;
an air outlet member with an air outlet; and
an air flow passageway placing the air inlet opening in communication with the air outlet,
wherein the air outlet member is configured in use to locate above the tongue, with the air outlet directing inhaled air between the tongue and the hard palate in a direction towards the posterior oral cavity.

The oral appliance body may be suitably manufactured from a soft and/or resilient material, suitably a thermoplastics material.

The oral appliance body, and in particular the arms may suitably have a degree of compliance such that the oral appliance may be fitted to patients with different sizes of dental arches.

The thermoplastics material may soften at temperatures below 100° such that it is user moldable. Such materials include ethylene vinyl acetate (EVA) that allows a user to soften the device in hot water and mold the device about the user's teeth as is well known with mouth guards. The oral apparatus may also have a core or frame from material that does not soften below 100° C. so as to provide structural support to the appliance.

Alternatively, the oral appliance may be formed from a soft thermoplastics material that does not soften below 100° soft polymer but is soft and compliable at room temperature such as polyurethane or silicone. This may be compared to known oral appliances such as the MADs that are made from injection molded ethylene vinyl acetate (EVA) or liquid silicon rubber (LSR). Silicone is particularly suitable as it is pliable and does not require molding to a user's teeth. This may improve comfort and allow the user some jaw movement that will also contribute to user comfort and thus compliance.

Suitably, the oral appliance may be made from silicone or other material by injection molding. Optionally, the described appliances may be injection molded from EVA, PU, and LSR.

The oral appliances may be made in several different stock sizes so that a substantial portion of the general population can select an appliance that can be fitted over their upper arch with a reasonable fit.

The oral appliance has an air inlet opening and an air outlet. As will be described below, the disclosed oral appliance delivers air into the oral cavity with minimal interference from the tongue. For this reason, the terms inlet and outlet are used to describe the air flow direction when inhaling. The opposite flow direction occurs when exhaling.

The oral appliance is U-shaped with an air inlet opening at the front so as to allow for mouth breathing. The air inlet may be any suitable shape or configuration. Suitably the air inlet has a cross-sectional area for breathing that allows a patient to breathe fully through the mouth. This may be important for patients with nasal obstruction. The oral appliance also allows a person to partially breathe through the nose.

In one embodiment, the air inlet opening may be formed in the front of the appliance body.

In another embodiment, the appliance may an air inlet member that extends forward of the front of the appliance body such that the air inlet opening is spaced from the body and the air inlet member forms part of the air flow passageway between the air inlet opening and the air outlet.

The air inlet member body may also define part of the air flow passageway.

Suitably the air inlet member is configured so as to allow a patient's lips to form a lip seal about the air inlet member. This prevents air from entering the oral cavity directly through the mouth rather than through the air inlet. In one aspect, the air inlet member has an elliptic cylindrical shape. It also helps to resist air from leaking out through the mouth.

In another aspect, the air inlet member has an elliptic frustoconical shape with side walls that taper from the air inlet opening towards the appliance body.

The oral appliance has an air outlet member with a body that in use locates above the tongue and directs inhaled air between the tongue and the hard palate towards the posterior of the oral cavity, and particularly the pharyngeal airway.

Suitably, the air outlet member is configured that in use the air outlet delivers air at a location along a line extending between the anterior teeth and the molar region.

Suitably, the air outlet member is configured to abut or be positioned close to the hard palate. Suitably, the air outlet member has a degree of reliance or resilience such that it resiliently contacts or presses against the hard palate. This may assist in directing air flow above the tongue during inhalation so as to avoid obstruction thereby. It may also create a seal with the hard palate so as to prevent or minimize air passing above the air inlet member during exhalation to assist in directing air into the air outlet.

Suitably, the air flow passageway between the air inlet and air outlet member includes a passageway part that is constricted with respect to the air outlet member.

A fluid flow through an abrupt constriction in a pipe can result in a decrease in pressure of the fluid. Fluid flow also experiences a decrease in pressure when travelling through a bend in a pipe. Fluid pressure also drops when fluid flows through an abrupt expansion. Whilst not wishing to be bound by theory, it is believed that the flow of air upon inhalation from the inlet member through the constricted part, bend in the constricted part and from the air outlet into the oral cavity experiences a decrease in pressure. This decrease in pressure results in a positive external pressure that may assist inhalation. Suitably, the oral cross sectional flow area of the air inlet member before the constricted part may be between about 200 mm² to about 400 mm². The cross-sectional flow area of the constricted part may be between about 50 mm² and about 100 mm².

Suitably, the oral appliance is configured to encourage the tongue into a forward position that corresponds to its natural resting position. When the tongue is in its natural resting position, the tip positions on the incisal papilla at the anterior part of the upper palate. With the tip of the tongue in this position, the dorsum of the tongue runs at the cervical third of the crowns and roots of the upper premolars. The base of the tongue goes downward at the molars, leading to its insert at the hyoid bone. When the tip of the tongue is in its physiological position, its dorsum and base tend to reposition at their physiological positions as well, with the base descending at the molar area. However, when mouth breathing, the tongue is in a lowered position to allow the patient to more easily breathe through the mouth. A disadvantage of this position is that the lowered tongue may partially block the airway.

The tip of the tongue is highly sensitive and haptically explores objects in the mouth and provides perception of size and shape of objects in the mouth. This is important for mastication and swallowing. The presence of the air outlet member in the oral cavity will generally evoke a spontaneous response for the tongue to find its way towards to touch and explore the air outlet member.

The arrangement of the air outlet member located above the tongue that directs air above the tongue allows the patient to inhale and exhale through the air inlet opening with the tongue in the elevated position.

The oral appliance may further comprise a lower dental arch receiving channel and the upper and lower dental arch receiving channels may be configured so that when the oral appliance is worn in the mouth, the patient's mandible is advanced. This further brings the tongue forward and may alleviate any obstruction of the pharyngeal airway.

Still further, air flow into the patient's mouth with the oral appliance generally bypasses saliva ducts so that mouth dryness is reduced or alleviated.

As discussed in the introductory section, there are a number of disadvantages with using conventional MAD devices. It is generally believed that the further the mandibular advancement, the more effective is the MAD device. Generally mandibular advancement of between about 7 mm to about 12 mm is recommended.

On the other hand, with the appliance in the present application, the tongue has already been voluntarily brought forward so that less physical mandibular advancement may be required. For example, mandibular displacement of about 5 mm or less or between about 1 mm to about 3 mm may be sufficient. It will be appreciated that this lesser degree of displacement, will provide less strain on the TMJ and less retractive action of the maxilla.

The web suitably thickens from the front of the web to a point towards the trailing ends of the arms. This tends to fill in the space between the teeth of the upper and lower jaw. This configuration in some respects resembles an airfoil, or aero foil, and thickens the web. This arrangement puts more pressure on the rear molars thereby relaxing and exercising the joints and muscles.

Suitably, the thickened portions of the web are compressible. Compression may be achieved by providing a section of softer or more compressible material. Suitably compression is achieved by providing one or more holes through the trailing ends of the arms of the web.

The combination of the aero foil shape and the ability to compress that part of the web between the rear molars can alleviate TMJ pain and other discomfort that is felt by users of conventional rigid devices. Further, the ability to compress the web allows movement of the user's jaws relative to each other, further alleviating discomfort.

The present disclosure also relates to a method of treating symptoms of SDB in a patient comprising the steps of:
providing an oral appliance as disclosed in all aspects herein; and
causing the patient to wear the oral appliance whilst sleeping.

In one form, the SBD is snoring. In another form, the SBD is OSA.

Suitably, the appliance is worn during sleep for a minimum of three hours during a night's sleep, suitably four hours, suitably five hours or more.

In one embodiment of the method, the oral appliance is used in combination with a CPAP machine in which the CPAP machine is connected to the air inlet opening of the oral appliance for blowing air into the mouth.

This is different to a conventional CPAP therapy in which air is blown into the nose. With the disclosed method, air can be introduced into the oral cavity. This allows patients with nasal congestion and/or obstruction who are incompatible with conventional CPAP to be able to use CPAP.

Further, the oral appliance is held in place by the teeth within the arch receiving channels and the seal is provided by the lips about either the air inlet member that forms part of the oral appliance that receives an air outlet adapter from the CPAP machine, or an air outlet adapter from the CPAP machine receives the air inlet member of the oral appliance.

In this way, the use of conventional CPAP masks may be avoided. The oral appliance bypasses the nasal cavity and any obstruction therein.

Also disclosed herein is a combination comprising an oral appliance as defined in any one of the preceding aspects of the disclosure, and an adapter having an adapter inlet or air inlet and an adapter outlet or air outlet and an adapter air passage between the adapter inlet and the adapter outlet, wherein the adapter outlet is configured for fluid communication with the air inlet opening of the appliance and the adapter inlet is configured for fluid communication with an air supply.

Further, as discussed above, the oral appliance locates the tongue in an elevated position bringing the mandible forward. Whilst not wishing to be bound by theory, it is believed that the combination of bringing the tongue and mandible forward may have a positive effect on the airway, thereby allowing CPAP to be applied with a lower pressure than would be required with a conventional CPAP interface.

A reduction in the pressure of CPAP may avoid or reduce undesirable side effects such as air in the stomach, stomach pain or gas. Still further, many patients find it difficult to exhale against the positive pressure of the CPAP and a reduction in the air pressure that is used may assist this.

Alternately the described device can also be used in conjunction with nasal CPAP, therefore positively ventilating through the nose and mouth while simultaneously advancing the mandible and the tongue. This combination (trilevel) positive ventilation would also require far less PAP and therefore less leakage would occur, there would be a minimal need for tight straps and air via oral ventilation will have minimal restriction from the tongue, as opposed to most MAD appliances which require substantial mandibular advancement accompanied by the detrimental effects previously stated.

According to yet another aspect of the invention there is provided a method of treating symptoms of SDB in a patient comprising:
providing a combination of an oral appliance and an adapter as defined in any preceding aspect of the invention, and connecting the adapter inlet to a CPAP machine; and
fitting the oral appliance to the patient, and causing air from to flow from the CPAP machine through the adapter air passage and the air flow passageway in the oral appliance, into the patient's mouth,
wherein the air flowing into the patient's mouth assists in opening the patient's airways while they are sleeping.

The adapter and the oral appliance may comprise any one or more of the features or combinations of features of the adapter and appliance defined in any other aspect of the invention.

According to yet another aspect of the invention there is provided a method of treating symptoms of SDB in a patient comprising:
providing an oral appliance as disclosed in any other aspect of the invention,
fluidly connecting the air inlet member to a CPAP machine; and
causing air to flow from the CPAP machine into the patient's mouth through the air inlet member, wherein the patient s the oral appliance whilst they are sleeping.

The adapter and the oral appliance may comprise any one or more of the features, or combination of features, of the adapter and appliance as defined in any other aspect of the invention.

According to a yet further aspect of the invention there is provided an oral appliance for the treatment of sleep disorder breathing in a patient, comprising:
an appliance body with a front section and two arms, the appliance body including an inner wall and an outer wall, a web interconnecting the inner wall and the outer wall so as to define an upper dental arch receiving channel;
an air inlet opening for intake of air; an air outlet member with an air outlet; and
an air flow passageway placing the air inlet opening in communication with the air outlet.

The oral appliance may comprise any one or more of the features or combination of features of the oral appliance as defined in any other aspect of the invention.

### BRIEF DESCRIPTION OF THE DRAWINGS

FIG. 1 is a top front perspective view of one aspect of an oral appliance as disclosed herein;

FIG. 2 is a rear top perspective view of the same oral appliance shown in FIG. 1;

FIG. 3 is a front view of the oral appliance shown in FIG. 1;

FIG. 4 is a rear view of the oral appliance shown in FIG. 1;

FIG. 5 is a side view of the oral appliance shown in FIG. 1;

FIG. 6 is a cross sectional view of the appliance shown in FIG. 1

FIG. 7 is a top plan view of the oral appliance shown in FIG. 1;

FIG. 8 is a bottom plan view of the oral appliance shown in FIG. 1;

FIG. 9 is a top plan view of an oral appliance of a further disclosed aspect;

FIG. 10 is a schematic top plan view of the oral appliance shown in FIG. 1 and the relationship with the positions of the teeth of a patient's upper arch;

FIG. 11 is a schematic view of tongue position relative to the oral appliance of FIG. 1;

FIG. 12 is a front perspective view of an oral appliance of a still further disclosed aspect;

FIG. 13 is a rear perspective view of the oral appliance as shown in Figure

FIG. 14 is a front view of the oral appliance as shown in FIG. 12;

FIG. 15 is a rear view of the oral appliance as shown in FIG. 12;

FIG. 16 is a side view of the oral appliance as shown in FIG. 12;

FIG. 17 is a cross section view of the oral appliance as shown in FIG. 12;

FIG. 18 is a top view of the oral appliance as shown in FIG. 12;

FIG. 19 is a bottom view of the oral appliance as shown in FIG. 12;

FIG. 20 is a cross section of a still further oral appliance as disclosed herein;

FIG. 21 is a front perspective view of a CPAP adapter for use with the disclosed oral appliances;

FIG. 22 is a rear perspective view of the adapter shown in FIG. 21;

FIG. 23 is right end view of the adapter shown in FIG. 21;

FIG. 24 is a left end view of the adapter shown in FIG. 21;

FIG. 25 is a side view of the adapter shown in FIG. 21;

FIG. 26 is a cross section view of the adapter shown in FIG. 21;

FIG. 27 is a top plan view of the adapter shown in FIG. 21;

FIG. 28 is a front perspective view of the oral appliance as shown in FIG. 12 with the adapter as shown in FIG. 21 fitted thereto;

FIG. 29 is a side view of the oral appliance adapted combination as shown in FIG. 28;

FIG. 30 is a cross section view of the oral appliance adapted combination as shown in FIG. 28;

FIG. 31 is a front perspective view of another embodiment of an oral appliance as disclosed herein;

FIG. 32 is a side view of the oral appliance as shown in FIG. 31;

FIG. 33 is a front perspective view of the oral appliance as shown in FIG. 33 with a CPAP adapter fitted thereto;

FIG. 34 is a side view of the oral appliance as shown in FIG. 33 with the adapted shown in cross section;

FIG. 35 is a front perspective view of an adapter in accordance with another embodiment of the invention for connecting an intra-oral appliance to a CPAP machine, in embodiments;

FIG. 36 is a rear perspective view of the adapter in FIG. 35;

FIG. 37 is a front view of the adapter in FIG. 35;

FIG. 38 is a top plan view of the adapter in FIG. 35;

FIG. 39 is a perspective view of the adapter in FIG. 35 showing a longitudinal section through the adapter;

FIG. 40 is a side view of the adapter shown in FIG. 35 engaged with an intra-oral appliance similar to that in FIGS. 1 to 20 to operatively connect them to each other;

FIG. 41 is a cross-sectional view through the adapter and intra-oral appliance in FIG. 40 showing the air outlet portion of the adapter received within the air inlet member of the appliance, in embodiments;

FIG. 42 is a schematic drawing showing the intra-oral appliance and adapter fitted to a patient with the intra oral appliance mounted over the mandibular and maxillary arches of the patient and showing the relevant anatomy of the patient, in embodiments;

FIG. 43 is a schematic perspective view of the intra-oral appliance and adapter fitted to a patient and showing their relationship with the facial features of the patient, in embodiments; and

FIG. 44 is a schematic side view showing the appliance and adapter fitted to a patient with the surrounding anatomy of the patient shown in section.

### DETAILED DESCRIPTION

An oral appliance and an apparatus in accordance with this disclosure may manifest itself in a variety of forms. It will be convenient to hereinafter describe several embodiments of the invention in detail with reference to the accompanying drawings. The purpose of providing this detailed description is to instruct persons having an interest in the subject matter of the invention how to carry the invention into practical effect. However, it is to be clearly understood that the specific nature of this detailed description does not supersede the generality of the preceding broad disclosure.

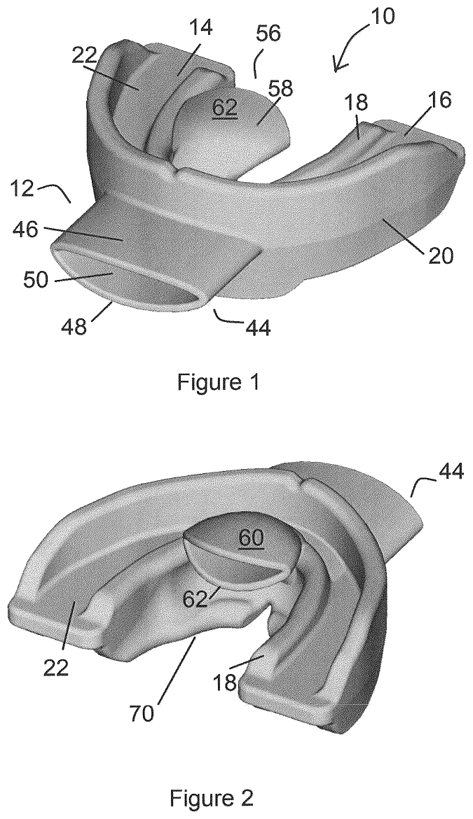

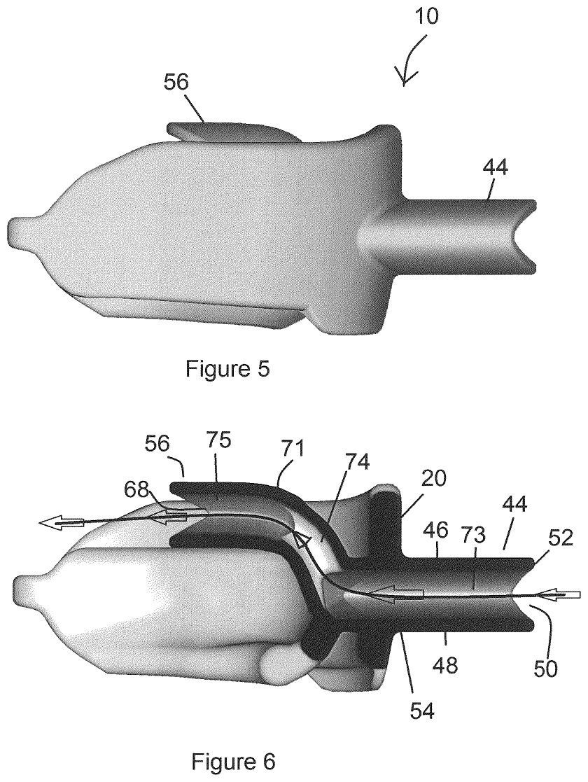

FIGS. 1 to 8 show a first aspect of an oral appliance 10. The appliance 10 may be made of medical grade silicone that is a rubber material that is flexible and comfortable in the mouth. The flexibility can also accommodate users having different oral dimensions such as arch sizes and arch shapes. The flexibility still further allows some movement that not only can provide comfort but can accommodate movement of the TMJ so as to reduce strain on the joint.

The appliance may also be dual molded comprising an internal base member that does not substantially soften at temperatures below 100° C., and an outer or surround layer of a thermoplastic material (such as ethylene vinyl acetate (EVA) that softens below 100° C.) extending over the base member. The base member may be a polyolefin such as polyethylene (PE), polypropylene (PP) or blends thereof. One example of a suitable polyolefin blend is a PE/polyurethane (PU) blend. The base member confers a suitable level of rigidity on the base member but does have some flexibility and this enables the appliance to accommodate arch forms of varying width. It will be appreciated that the flexibility allows for better fit whilst the moldable outer layer provides a degree of customization.

The appliance 10 includes a U-shaped appliance body for mounting over the upper arch of a user. The appliance body has a front section 12 and two arms 14, 16. The appliance 10 includes an inner wall 18 that is positioned on a lingual side of the patient's upper arch and an outer wall 20 that is positioned on the buccal side thereof. The outer wall 20 is U-shaped with a front 20f and two arms 20a. The inner wall 18 is U-shaped with a front 18f and two arms 18a. The appliance 10 also includes a U-shaped web 22 interconnecting the inner wall 18 and the outer wall 20. When the appliance 10 is in the mouth of a user, the web 22 lies in the occlusal plane between the dentition of the upper and the lower arches in use.

The inner 18, outer wall 20 and web 22 define upper 24 and lower 26 arch receiving channels within which respectively the upper arch and associated dentition, and the lower arch and associated dentition can be received (most clearly seen in FIG. 4).

The inner wall 18 includes an upper portion 28 which projects up from the web 22 and a lower portion 30 which projects down from the web 22. Similarly, the outer wall 20 comprises an upper portion 32 above the web 22 and a lower portion 34 below the web 22. The inner wall 18 has a lingual surface 36 and a channel surface 38. The outer wall 20 has a front buccal surface 40 that is dimensioned so that it substantially covers the buccal aspects of the upper and lower posterior teeth.

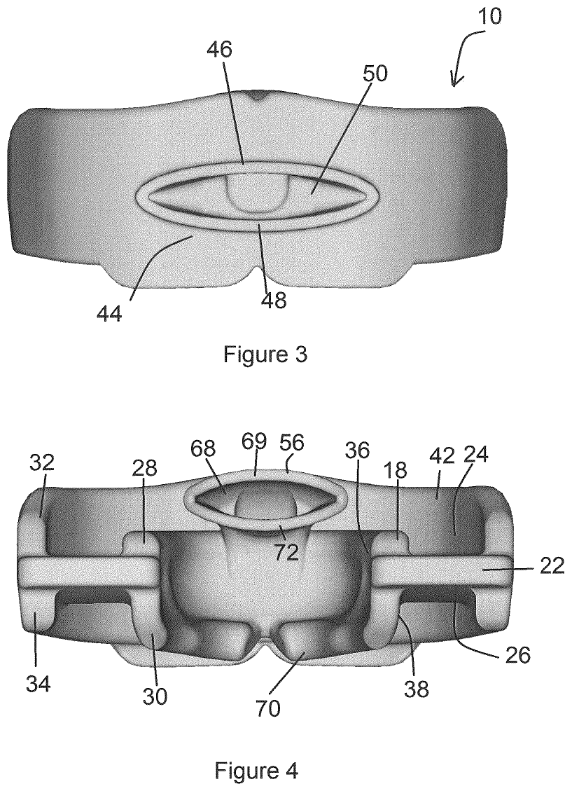

The oral appliance 10 includes an air inlet member 44 extending forwardly from the front buccal surface 40 of the outer wall 20. The air inlet member 44 may be any suitable shape or configuration and for example may be elliptical or ovoid in cross section. The air inlet member 44 is located generally centrally on the buccal surface 40. The air inlet member 44 has a curved upper wall 46 and a curved lower wall 48 and an elliptical inlet 50 (as can be seen in FIG. 3). The inlet 50 is surrounded by a lip 52 (as seen in FIG. 6). In another aspect, the air inlet may be ovoid. The air inlet may also comprise a plurality of inlet apertures.

The curvature of the upper and lower walls 46, 48 of the air inlet member 44 is designed for a user's lips to fit comfortably around so as to be able to form a lip-seal around the air inlet member 44. This means that the only way that air can pass into the mouth is through the air inlet 50. It will be appreciated that other shapes or configurations of the air inlet member may be suitable for the purpose. As can be seen in the cross section in FIG. 6, the upper wall 46 and lower wall 48 define an air passage 54 that extends through the outer wall 20.

The frontal portion of the inner wall 18f inclines rearwardly away from the outer wall 20 as it extends up from the web 22 at an angle of about 30 to 40 degrees. This matches the curvature of the lingual side of the maxillary teeth. The lower portion of the inner wall 18 includes a tongue elevator 70 (shown in FIGS. 2 and 4). The lower part 181 of the inner wall 18 has a lower terminal edge region that is thickened to form the tongue elevator 70. The tongue elevator 70 forces the tongue to hold an upper position that further assists in bringing the tongue forward so as to open the airway.

The upper part 28 of the inner wall 18 comprises a rearward facing extended part that forms an air outlet member 56 in a substantially central position. The air outlet member 56 has an outlet body 58. The outlet body 58 has an upper wall 60 that is concave in the transverse direction and a lower wall 62 that is also concave in the transverse direction.

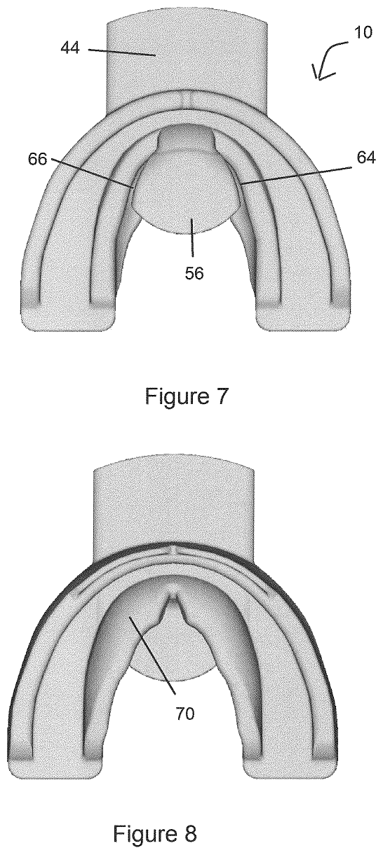

The outlet body 58 further comprises opposing side edges 64, 66 and a posteriorly facing elliptical air outlet 68. As seen in FIG. 7, the side edges 64, 66 of the outlet body 58 diverge outwardly towards the air outlet 68. The elliptical outlet 68 has an upper edge 69 that is convex in the transverse direction and a lower edge 72 that is convex in the transverse direction. The outlet body 58 is also arcuate about a radial center along the longitudinal axis of the body.

The air outlet member 56 is configured such that in use the air outlet 68 locates horizontally adjacent or close to the hard palate at a position between the molars, suitably in the region of the second premolar PM and first molar M as shown in FIG. 10. This places the air outlet 68 above the tongue about the middle part of the hard palate. The curvature of the upper wall 60 of the air outlet body 58 substantially corresponds to that of a user's hard palate for comfort.

The air outlet member 56 has a degree of resilience such that it may comfortably press against the patient's palate so as to prevent or obstruct air from passing above the air outlet member 56 and the palate during exhalation so as to optimize air flowing into the air outlet 68. The air outlet 68 is configured to extend substantially across the width of the hard palate at that point. In this way air passing out through the air outlet 68 enters the oral cavity and is spread uniformly across the width of the oral cavity.

An air flow passageway 71 is defined between the air inlet 50 and the air outlet 68. The air flow passageway 71 has an inlet part 73 defined by the air inlet member and an outlet part 75 defined by the air outlet member body 58. The oral appliance 10 includes a curved intermediate part of 74 of constant cross section. The curved intermediate part 74 has an inner facing part and an outer part. The inner part receives the tip of a user's tongue. The tongue is highly responsive to objects in the mouth and will haptically explore the air outlet member 56 and this brings the tongue forward. Further advantages of this tongue placement will be described below.

The diameter of the curved intermediate part 74 is constant and about 6 mm to 8 mm. By contrast the width of the air inlet is about 23 mm to 26 mm.

Fluid flow through an abrupt constriction in a pipe results in a decrease in pressure of the fluid. Fluid flow also experiences a decrease in pressure when travelling through a bend in a pipe. Fluid pressure also drops when fluid flows through an abrupt expansion. Whilst not wishing to be bound by theory, it is believed that the flow of air upon inhalation from the inlet member through the constriction in intermediate part 74 and the bend therein, leads to a decrease in pressure that results in a positive external pressure that may assist inhalation.

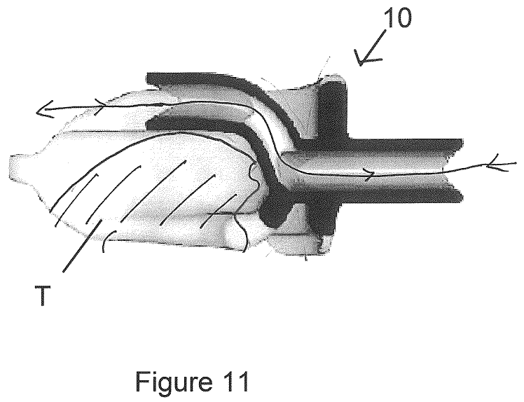

As discussed above, the oral appliance 10 encourages the tongue forward adjacent the air outlet member 56. This corresponds to the normal rest position for nasal breathing (subject to the thickness of the air outlet member). Under normal circumstances, such a tongue position would inhibit mouth breathing. With the present appliance, air is delivered above the tongue at a position about halfway along the hard palate and bypasses any such inhibition, whilst encouraging a forward location of the tongue T. This is schematically shown in FIG. 11. The air outlet member 56 is also flexible that allows the tongue to press up against it. A patient may be encouraged to do this to exercise and train the tongue to adopt the correct position, when the appliance is not being worn.

Whilst not wishing to be bound by theory it is believed that the positive pressure delivered to the mouth through the air outlet member 56 combined with shape and configuration of upper part of the air outlet member against the hard palate and further the activity of the tongue against the air outlet member 56 may also assist in the expansion of the maxillary arch. The lateral wall of each nasal cavity is mainly formed by the bone of the maxilla. Thus, an expansion of the maxillary arch may increase the size or width of the nasal cavity, thereby reducing nasal obstruction. Over time this may assist in alleviating at least partially the symptoms of OSA.

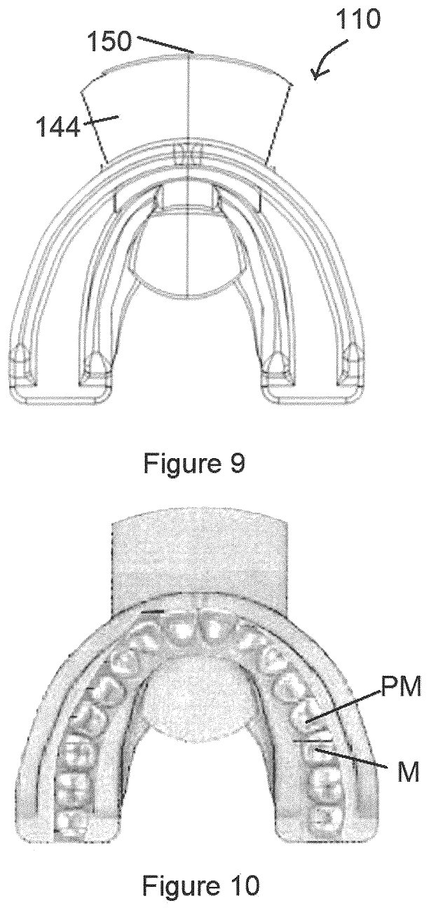

FIG. 9 shows an alternative oral appliance 110 in which the air inlet member 144 is tapered from the air inlet opening 150 towards the appliance 110. Further the upper and lower arch receiving channels are configured for mandibular advancement (see FIG. 6). This physically brings the mandible and tongue forward and further opens the pharyngeal airway.

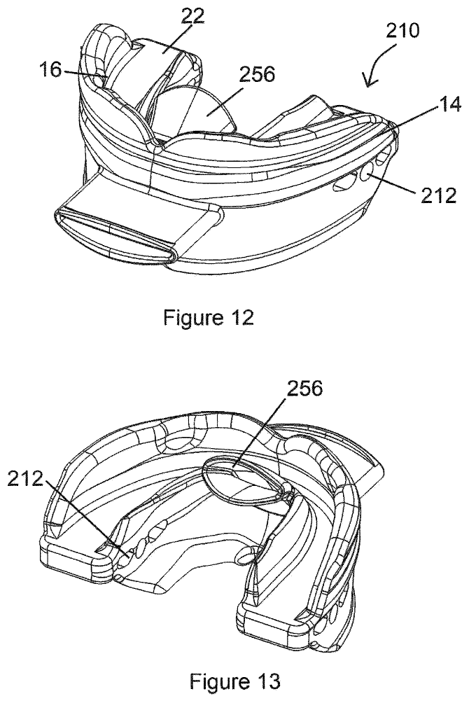

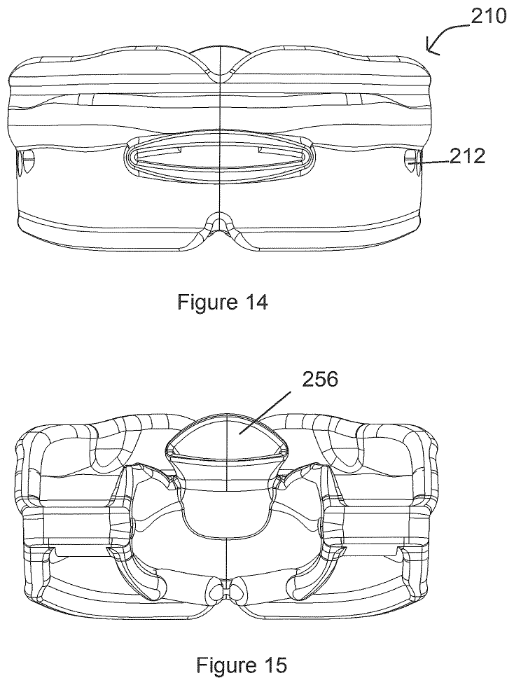

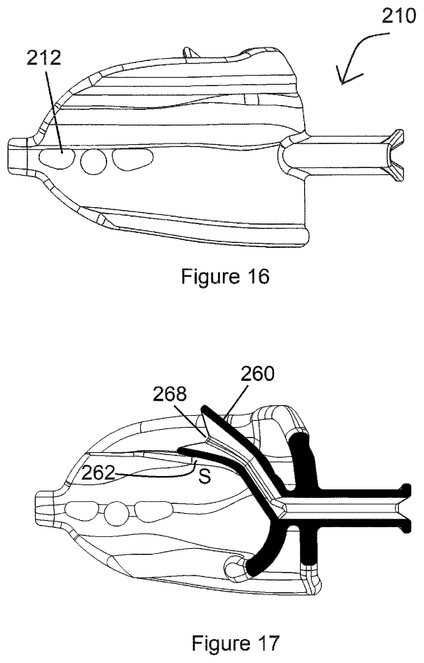

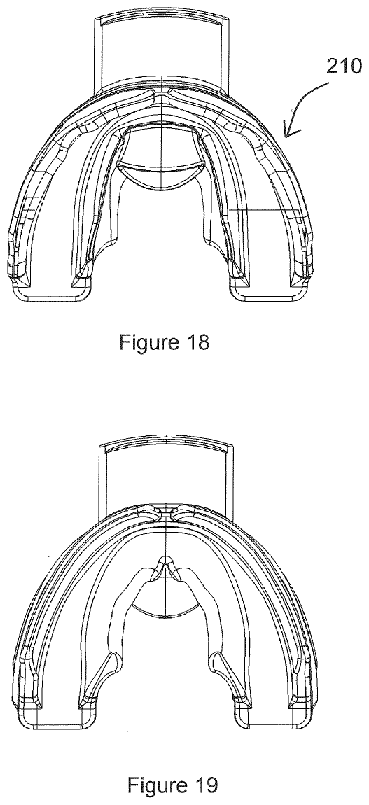

FIGS. 12 to 19 show an oral appliance 210 in accordance with another embodiment of the disclosure. The same reference numbers will be used to define the same features as in the earlier embodiment.

The oral appliance 210 is substantially the same as the oral appliance of FIG. 1 with the addition of three compressible channels 212 in the web 22 of each arm 14, 16. The channels 212 may be open or closed. The channels 212 are compressible that further allows relative movement of the jaw so as to reduce strain on the TMJ.

The air outlet member body 256 and the air outlet 268 each have a different configuration that may best be seen by comparing the cross-sectional views in FIGS. 11 and 17. The air outlet member body 256 has an upper wall 260 and a lower wall 262 that are diverging rather than parallel. The air outlet member body 256 is also at an angle of about 30 to 45 degrees to the horizontal rather than parallel. The air outlet body 256 is shorter and extends back to the premolars. The space where the tip of the tongue sits is larger than that in FIG. 11 and that may further facilitate correct tongue placement.

In FIGS. 12 to 20 the outer wall of the appliance body is more extensive and has upper and lower edges that extend beyond the teeth and over the gums of the patient in use. That is the outer wall is configured to extend higher and also extend lower than the outer wall on other appliances.

This increased height or vertical extent of the outer wall is quite apparent when the appliance in these figures is compared with that shown in FIG. 1 and compared with other intra oral appliances used for treating malocclusions. This increased height of the outer wall helps to resist the flow or leakage of air out of the mouth when positive air pressure is applied. Further the outer wall has an upper edge region and a lower edge region. These upper and lower edge regions are thickened relative to the remainder of the outer wall. This feature of thickening the upper and lower edge regions helps to resist the flow or leakage of air out of the mouth when positive air pressure is applied.

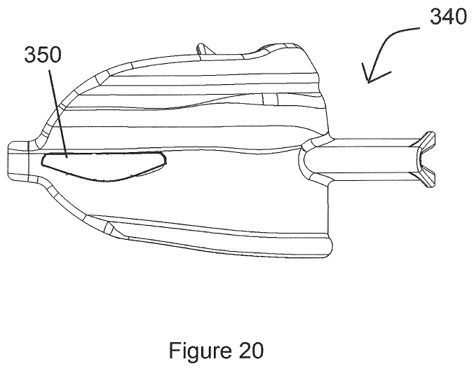

FIG. 20 shows yet a further oral appliance 340 similar to that of FIGS. 12 to 19 but having a singular air channel 350.

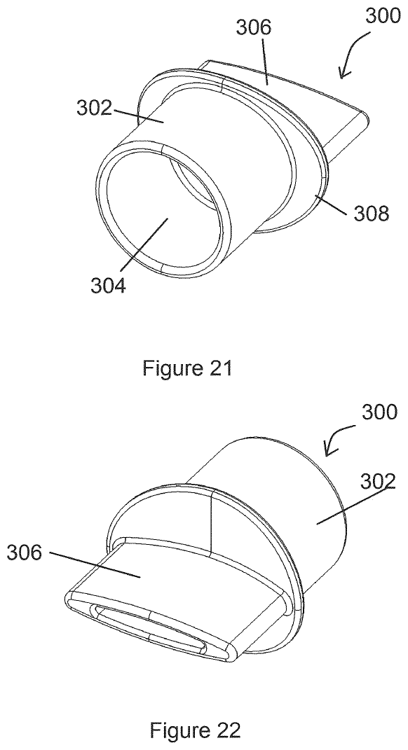

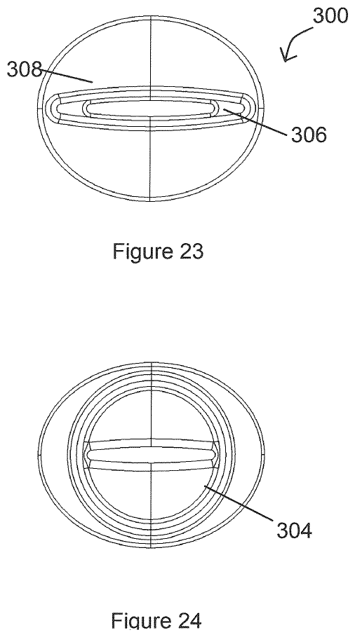

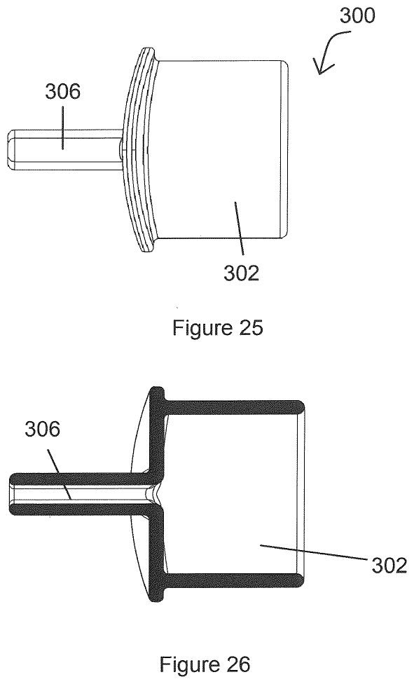

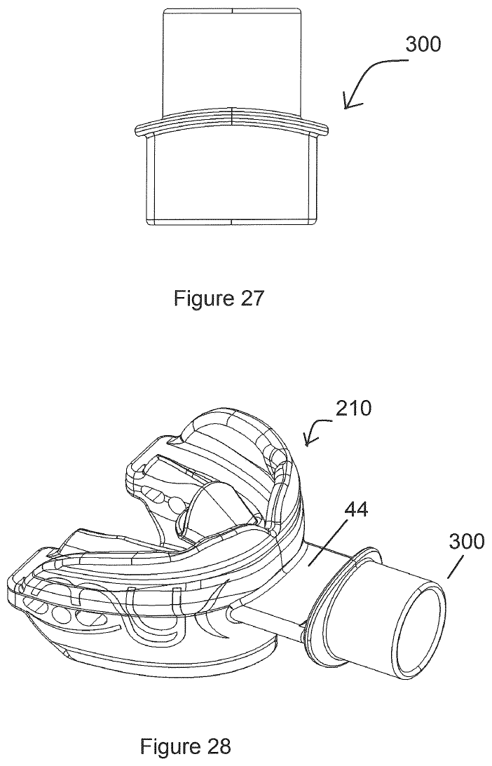

FIGS. 21 to 26 show an adapter 300 that may be used to connect the oral appliance to an outlet tube of a CPAP machine.

The adapter 300 has cylindrical air inlet member 302 with an inlet 304. The inlet member 302 is configured for fluid connection to a CPAP air outlet tube. The inlet member 302 may receive or be received by the CPAP tube. The adapter 300 has an outlet member 306 of elliptical cross-section that is configured for fluid connection to the air inlet member 44 of the oral appliances 10, 210. The air inlet member 44 may receive or be received by the air outlet member 306. The inlet member 302 and outlet member 306 are separated by an elliptical flange 308 that in use acts as a stop against a patient's lips.

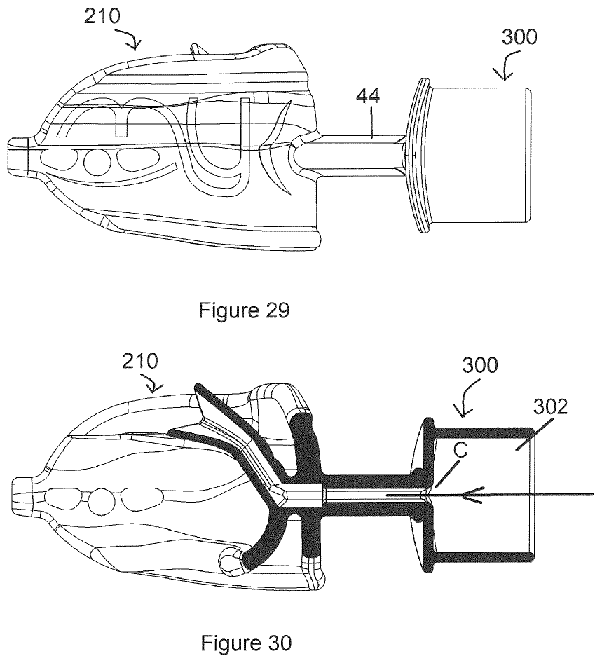

FIGS. 28 to 30 show the adapter 300 connected to the appliance 210. It will be appreciated that such an arrangement may be more comfortable for a user than a nasal of full face mask. Seals against the face and chin straps are not required.

As can be seen in FIG. 30 there is a sudden contraction C in the in-flow direction from the air inlet member 302 to the air outlet member 306. Sudden contractions in pipes cause a marked drop in pressure due to an increase in velocity and loss of energy to turbulence.

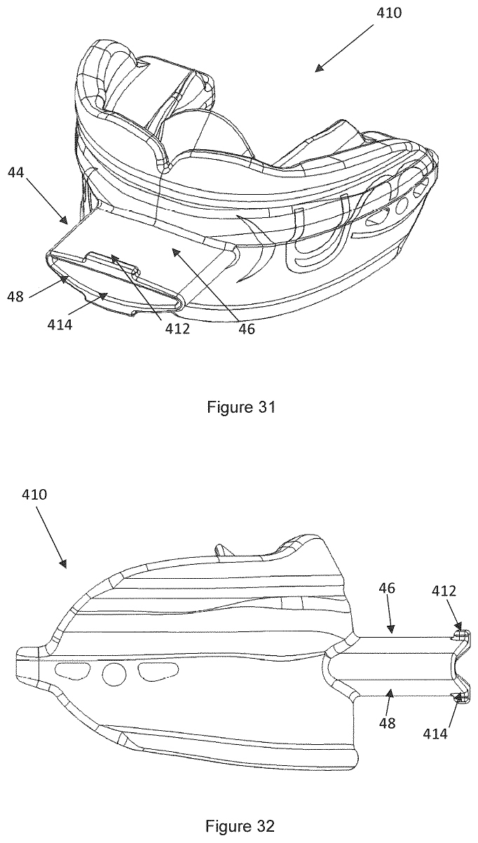

FIGS. 31 and 32 show an oral appliance 410 in accordance with another embodiment of the disclosure. The same reference numbers will be used to define the same features.

The oral appliance 410 is substantially the same as the oral appliance of FIG. 1 with the addition of an upper tab 412 formed on the upper wall 46 of the air inlet 44, and a lower tab 414 formed on the lower wall 48 of the air inlet 44.

In FIGS. 28 to 34 (as in FIGS. 12 to 20) the outer wall of the appliance body is more extensive and has upper and lower edges that extend beyond the teeth and over the gums of the patient in use. Further, the outer wall has upper and lower edge regions that are thickened relative to the remainder of the outer wall to help resist the flow or leakage of air out of the mouth when positive air pressure is applied.

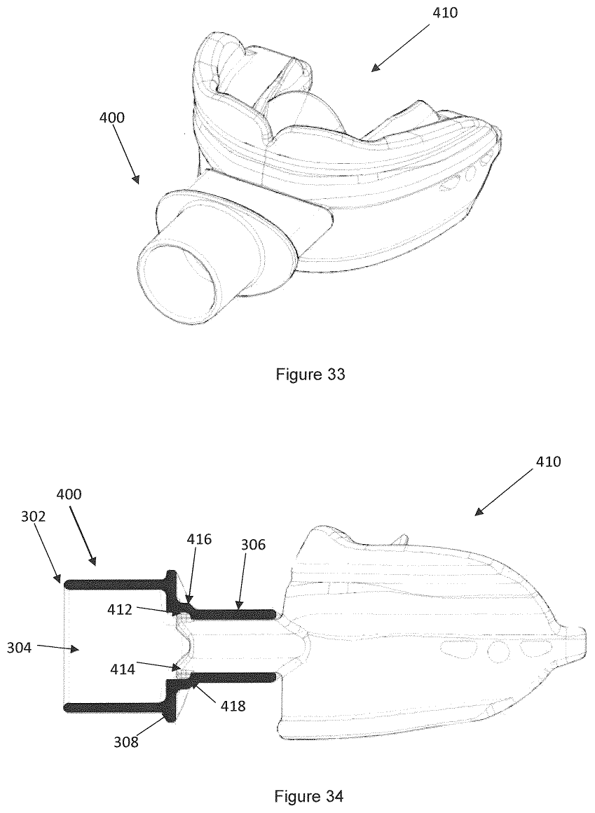

FIGS. 33 to 34 show an adapter 400 of another aspect connected to the appliance 410. In these figures, the same reference numbers will be used to define the same features unless otherwise specified.

The adapter 400 is substantially the same as the adapter of FIGS. 21 to 26 with the addition of an upper groove 416 and a lower groove 418 formed on an internal surface of the outlet member 306, positioned adjacent to the elliptical flange 308. The upper recess 416 is configured to engage with the upper tab 412 of the oral appliance and the lower recess 428 is configured to engage with the lower tab 414 to thereby releasably secure the air inlet member 306 of the adapter 400 to the appliance 410. The walls of the adapter are sufficiently resilient so as to provide for a snap fit engagement with the appliance 410.

A mandibular advancement for a person having a normal bite will put some strain on the temporomandibular joints (TMJ). However, with this appliance the strain is significantly less than with prior art known MAD devices. The flexibility of the silicone material or the material of a base member where the oral appliance is dual molded allows some relative movement of the mandible that may at least partially alleviate such strain. Common side effects of art known MAD devices include TMJ discomfort or pain and myofascial pain. Long term mandibular advancement can cause TMJ damage and dysfunction.

The combination of a forward tongue position and a minimal mandibular advancement that is achieved with the present appliances allow the pharyngeal airway to open to an extent comparable with known prior art MADs that significantly advance the mandible. Thus, there is less strain on the TMJ with use of the present appliances than with art known MADs. This allows for more patient comfort which generally translates to higher patient compliance. This location of the tongue also reciprocates retrusive action of the maxilla.

The intra-oral appliance may also be used in conjunction with a CPAP machine whereby the CPAP air tube is connected to the air outlet member.

It will be appreciated that the disclosed intra-oral appliances do not require custom fabrication fitting by virtue of the flexibility of the thermoplastic's material. The oral appliances may be easily manufactured by injection molding at a fraction of the cost of custom made devices. Alternately, the dual molded ready-made device can be custom fitted with the above features.

The configuration of the air outlet member promotes voluntary forward positioning of the tongue that opens the airway. This may reduce the amount of mandibular advancement required. This in turn reduces stress on the TMJ. Stress on the TMJ is further reduced by the flexibility of the appliance.

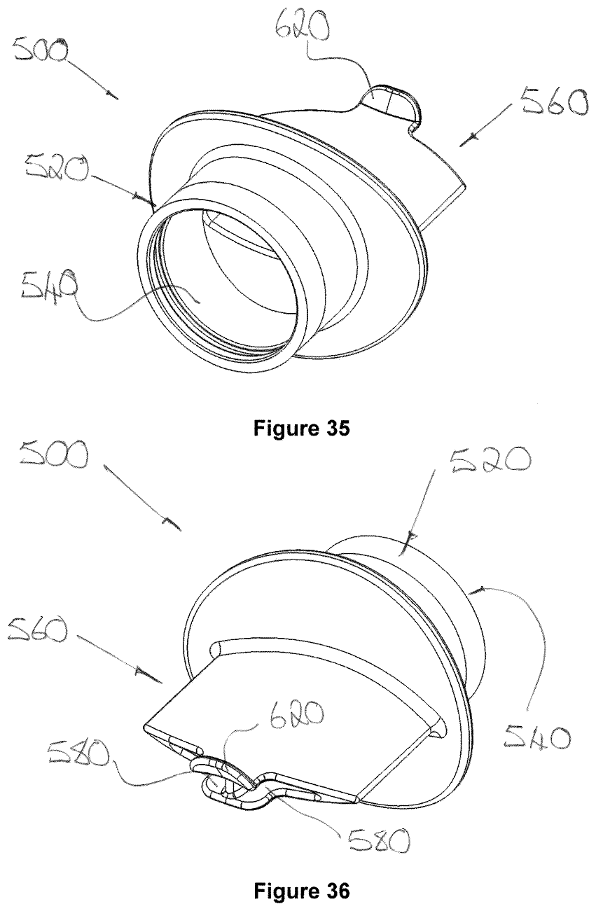

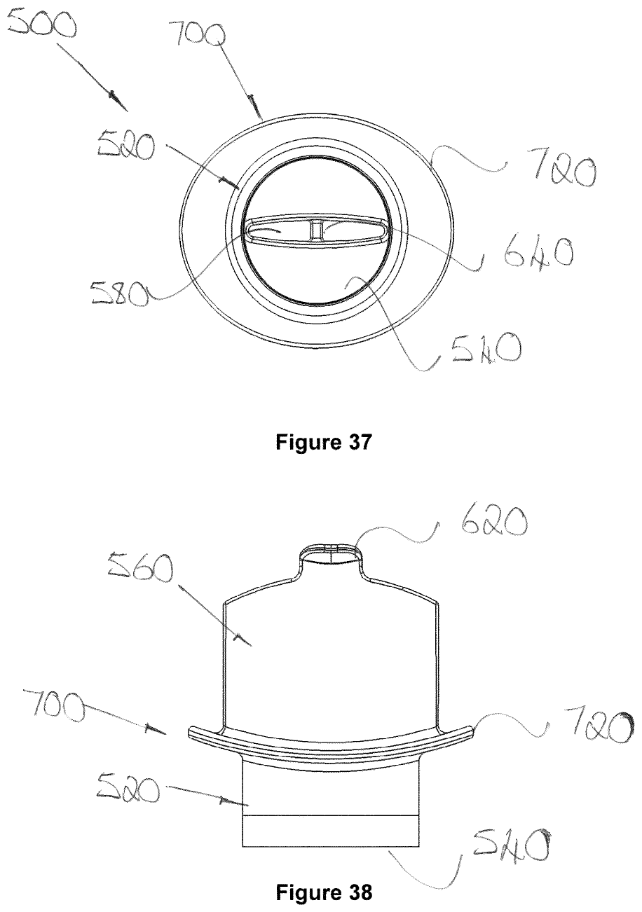

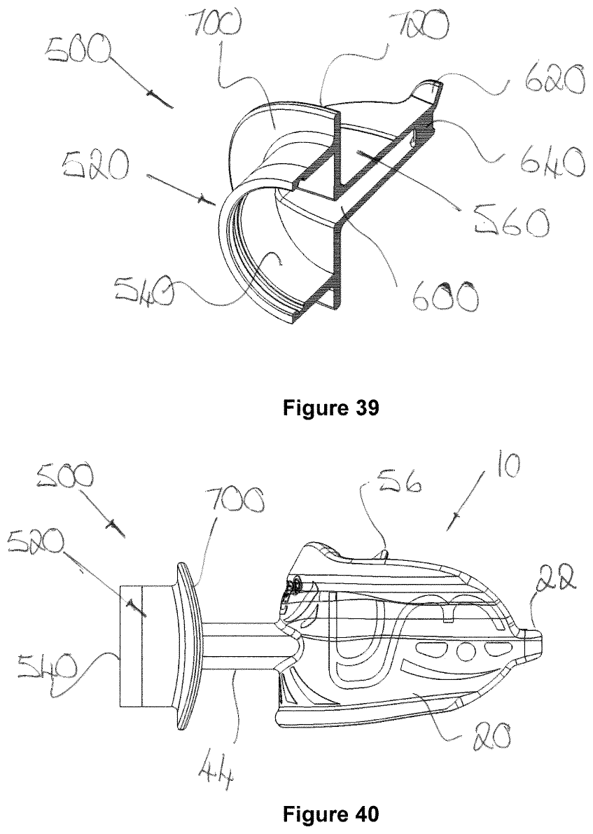

FIGS. 35 to 40 illustrate an adapter in accordance with another embodiment of the invention for use with an intra-oral appliance to provide an apparatus for connecting a CPAP machine to the intra-oral appliance for delivering CPAP treatment to the patient. In the drawings the adapter will be indicated generally by the reference numeral 500 and the features of the adapter will be described in more detail below.

Broadly, the adapter 500 comprises an air inlet portion 520 forming an air inlet 540 and an air outlet portion 560 forming an air outlet 580. The adapter 500 further comprises an air passage 600 extending from the air inlet 540 to the air outlet 580 so that air can flow as a stream through the adapter 500. The air outlet portion 560 is sized and configured to complement and be received within the air inlet member 44 of the intra-oral appliance 10 in use. The air inlet portion 520 is operatively connected to an outlet of an air tube or air hose of the connector in use which will be described in more detail below.

The air outlet portion 560 further comprises a catch formation or engagement formation 620 for releasably engaging the air inlet member 44 of the intra-oral appliance 10 and holding the adapter 500 to the intra-oral appliance 10. In the illustrated embodiment, the catch formation 620 on the outlet portion 560 comprises a lip that extends around an end of the air inlet member 44 of the appliance 10.

Further, the air outlet portion 560 of the adapter 500 comprises an internal passage support 640 for holding the air passage 600 in the outlet portion 560 fully open during use of the adapter 500. It resists the air outlet portion 560 from collapsing inward when it is received within a resilient air inlet member of the appliance 10. The internal passage support 640 therefore also holds the air inlet opening of the appliance open during use.

The adapter 500 further comprises an adapter flange or wall 700 extending transversely or radially out to a peripheral edge 720 of the wall 700 beyond the radial periphery of the air inlet portion 520 and the air outlet portion 560. The flange or wall 700 is axially positioned between the two portions 520 and 560. The flange 700 is also curved to complement the curvature on the person's face and mouth so that it can lie up against or close to an outer surface of the part of the patient's face around their mouth. This contributes to the performance of the adapter 500 by helping to resist air flow out through the mouth of the patient.

The adapter 500 is made of a substantially rigid material, e.g. a rigid polymeric material and the adapter as a whole is substantially rigid.

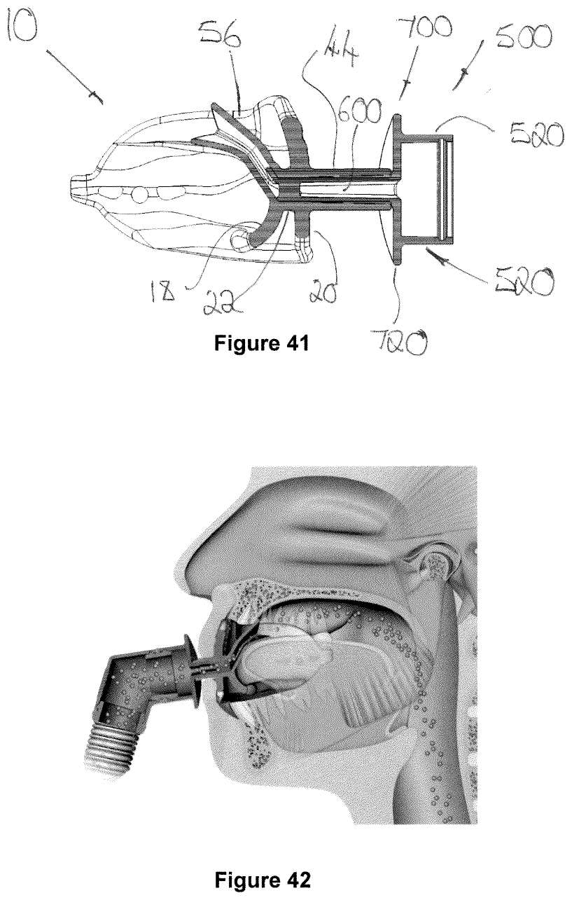

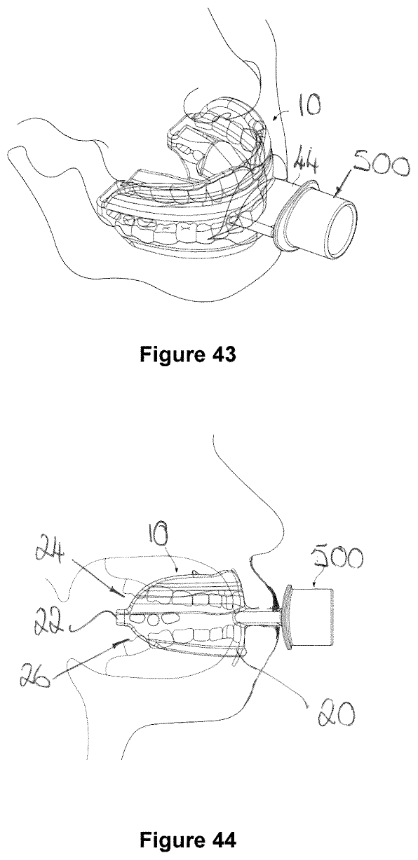

The intra-oral appliance shown in FIGS. 40 to 44 is broadly similar to the appliance shown in FIGS. 1 to 8 with some differences that are discussed below. Accordingly, the same reference numerals will be used to refer to the same components unless otherwise indicated.

Broadly, the intra-oral appliance 10 comprises an appliance body forming an upper arch receiving channel 24 and a lower arch receiving channel 26. The appliance body in turn comprises an outer buccal wall 20 and an inner lingual wall 18 and a web 22 joining the lingual and buccal walls 18 and 20. The web 22 extends broadly in the occlusal plane.

The intra oral appliance 10 comprises an air inlet member 44 forming an air inlet opening 50 extending forward from the appliance body. In the illustrated embodiment, the air inlet member 44 is formed of a resilient and deformable material that can be deformed in use. As the intra oral appliance 10 and in particular the air inlet member 44 thereof is quite resiliently flexible, it can be easily deformed with little pressure. Therefore, the rigid air outlet portion 560 of the adapter 500 that is received within the air inlet member 44 of the appliance 10 assists in holding the outlet member open (and support it against collapsing) so that air can flow therethrough.

The illustrated air inlet member 44 has a substantially constant elliptical cross-sectional configuration along its length that is sized and configured to enable the patient's lips to form a seal about the air inlet member 44.

The intra oral appliance 10 also comprises an air outlet member 56 forming an air outlet opening 68 configured to locate above the patient's tongue in use, and an air flow passageway 71 between the air inlet opening 50 and air outlet opening 68 to place the and outlet in fluid communication.

The air outlet member 56 may be configured to abut or be close to the patient's hard palate and be formed of a resilient material that can be deformed in use into contact with the hard palate. The air outlet opening 68 in the air outlet member 56 has a substantially elliptical configuration. Further, the air outlet member 56 may taper outwardly along its length in a direction towards the air outlet opening 68.

In use, the combination of the appliance 10 and the adapter 500 is placed in a patient's mouth with the upper and lower arch channels 24 and 26 respectively of the appliance 10 fitted over the maxillary and mandibular arches respectively of the patient. The air inlet member 44 of the appliance 10 projects forwardly out of the patient's mouth between the lips of the patient. FIG. 42 shows the intra-oral appliance 10 and adapter 500 fitted to a patient with the intra oral appliance 10 mounted over the mandibular and maxillary arches of the patient.

The adapter 500 is operatively connected to the air inlet member 44 of the appliance 10 by inserting the air outlet portion 560 of the adapter 500 through the air inlet opening 50 into the air inlet member 44 of the appliance 10. Thus, the air outlet portion 560 of the adapter 500 is received within the air inlet member 44 which circumferentially surrounds it.

The air outlet portion 560 has a flattened substantially elliptical configuration that complements the air inlet member 44 of the appliance 10 and enables it to be snugly inserted into the air inlet member 44 through the air inlet opening 50. When the air outlet portion 560 is fully inserted, the catch formation 620 on the adapter 500 passes over a free outer or terminal end of the air inlet member 44 (spaced from the appliance body) and holds it in position engaged with the air outlet portion 560. Further, the support posts 640 within the air outlet portion 560 support it in an open position that permits the passage of air therethrough.

It will be appreciated that the rigid air outlet portion 560 of the adapter 500 holds the resilient air inlet member 44 open during use. The support post 640 in particular has been very useful for holding the air passage 600 open.

The outer wall 20 of the appliance body and the adapter wall 700 may be configured to be positioned on respectively inner and outer sides of the patient's lips in use and help to resist the leakage of air out through a mouth opening of the patient, e.g. during the application of positive air pressure. As shown in one or more of the drawings, each of the outer wall 20 and the adapter wall 700 may cover at least part of the patient's mouth. This helps to resist the leakage of air out of the mouth opening when air is passed through the intra-oral appliance and into the mouth of the patient during treatment by the application of positive air pressure.

An air tube (not shown) is connected to the air inlet portion 520 of the adapter 500 and leads away from the mouth of the patient or indirect coupling to a CPAP machine (not shown). Conveniently, the air tube may comprise a lightweight flexible hose with a helical spiral along its length. In one form, the hose is made of a transparent polymeric material such as polyethylene.

During treatment, a CPAP machine supplies continuous positive air pressure (CPAP) by blowing air through the inlet portion 520 of the adapter 500 and then out through the outlet portion 560 of the adapter 500 into the air inlet member 44 of the intra-oral appliance 10. From there, the air is displaced through the air flow passageway 71 in the oral appliance 10 and out through the air outlet opening 68 in the air outlet member 56. This blows air into the patient's buccal cavity above the surface of their tongue and in a direction towards their oro-pharyngeal passage or cavity. This air pressure helps to resist the oro-and naso-pharyngeal passages of the patient from collapsing and keep them open for air flow. The Applicant has found that the arrangement described above works satisfactorily and may not have significant leakage of air out through the nostrils of the patient or out through the mouth of the patient (known as mouth leak).

Typical CPAP treatment protocols may be used with the Applicant's system. However, Applicant believes that its system and apparatus may enable a lower CPAP pressure to be used for treatment, and it still be effective when compared with treatment with nasal masks and face masks.

An advantage of the adapter and appliance described above with reference to the drawings is that it enables CPAP to be delivered through the mouth of the patient by means of a basic intra-oral appliance. Applicant believes this treatment will be more comfortable than a conventional CPAP treatment with a nasal mask particularly when it is worn overnight while a patient is sleeping. Further, it does not have the side effects of this CPAP treatment. Further, Applicant also believes that this system has the potential to deliver improved clinical outcomes over conventional CPAP treatment.

An advantage of the adapter described above with reference to the drawings is that it is received within the air inlet member of the appliance and acts to hold it open during use. Thus, it resists the air inlet member from collapsing inward and blocking air flow. Another advantage of the adapter is that it provides a catch for positively engaging an end of the air inlet member of the appliance and holding it operatively connected to the adapter. Applicant has found that the catch works well in practice to operatively connect the adapter and appliance to each other and provide unobstructed air flow.

In this specification, the term “comprising” is intended to denote the inclusion of a stated integer or integers, but not necessarily the exclusion of any other integer, depending on the context in which that term is used. This applies also to variants of that term such as “comprise” or “comprises”.

In the specification and claims, the term “sleep disorder breathing” (SBD) may refer to any condition where there is an abnormal breathing pattern during sleep. In some cases, this abnormality is because of an upper airway obstruction during sleep, including but not limited to include snoring, upper airway resistance syndrome (UARS), and obstructive sleep apnea-hypopnea (OSA). Abnormal breathing can also occur in the absence of any airway obstruction during sleep in which the patient stops breathing for a period of time, known as an apneic event.

It is intended that all matter contained in the above description or shown in the accompanying drawings shall be interpreted as illustrative only and not limiting. All such modifications and variations thereto, as would be apparent to persons skilled in the art, are deemed to fall within the broad scope and ambit of the invention as is set forth herein. Changes in detail or structure may be made without departing from the basic elements of the invention as defined in the following claims.

1. An adapter for connecting an intra-oral appliance on a patient to a CPAP machine,
the adapter comprising:
an air inlet portion forming an air inlet and an air outlet portion forming an air outlet, and an air passage extending from the air inlet to the air outlet,
wherein the air outlet portion has a complementary configuration to an air inlet member of the intra-oral appliance and is sized to be received within the air inlet member in use; and
the air inlet portion is operatively connected to an outlet of an air tube for connection to the CPAP machine.

2. The adapter for connecting an intra-oral appliance to a CPAP machine according to claim 1, wherein the air outlet portion comprises a curved upper major surface and a curved lower major surface, and the upper and lower major surfaces are joined along their side edges.

3. The adapter for connecting an intra-oral appliance to a CPAP machine according to claim 1, wherein the air outlet portion has a flattened substantially elliptical configuration broadly corresponding to a shape of the patient's mouth.

4. The adapter for connecting an intra-oral appliance to a CPAP machine according to claim 1, wherein the air outlet portion comprises an engagement formation or catch formation for releasably engaging the air inlet member of the intra-oral appliance and holding the adapter to the intra-oral appliance.

5. The adapter for connecting an intra-oral appliance to a CPAP machine according to claim 4, wherein the engaging formation is positioned at a terminal end of the air outlet portion, and is configured to sit behind an inner end of the air inlet member of the intra-oral appliance in use when the appliance is engaged with the adapter.

6. The adapter for connecting an intra-oral appliance to a CPAP machine according to claim 1, wherein the air outlet portion further comprises an internal passage support for holding the air passage open during use of the adapter.

7. The adapter for connecting an intra-oral appliance to a CPAP machine according to claim 6, wherein the internal passage support comprises a support post located in the air passage and extending across the air passage adjacent to the air outlet.

8. The adapter for connecting an intra-oral appliance to a CPAP machine according to claim 1, wherein the adapter further comprises an adapter wall extending transversely outward beyond a periphery of the air inlet portion and air outlet portion, and the adapter wall is axially positioned between the air inlet and air outlet portions.

9. The adapter for connecting an intra-oral appliance to a CPAP machine according to claim 8, wherein the adapter wall has a curved configuration for complementing a curvature of a patient's mouth and face.

10. The adapter for connecting an intra-oral appliance to a CPAP machine according to claim 8, wherein the adapter wall has a substantially elliptical configuration, and the adapter wall is elongated in the direction of the upper and lower major surfaces of the air outlet portion.

11. An apparatus for carrying out CPAP treatment, comprising:
an intra-oral appliance comprising an appliance body forming an upper arch receiving channel, and an air inlet member forming an air inlet extending forward from the appliance body, an air outlet member forming an air outlet opening configured to locate above the patient's tongue in use, and an air flow passageway extending between the air inlet member and the air outlet member for placing the air inlet opening and air outlet in fluid communication; and
an adapter mounted on the intra-oral appliance, the adapter comprising: an air inlet portion forming an air inlet and an air outlet portion forming an air outlet, and an air passage extending from the air inlet to the air outlet, wherein the air outlet portion has a complementary configuration to the air inlet member of the intra-oral appliance and is received therein, and the air inlet portion is operatively connected to an outlet of an air tube for connection to a CPAP machine.

12. The apparatus according to claim 11, wherein the air outlet portion of the adapter is substantially rigid, and the air outlet portion has a substantially elliptical cross-sectional configuration that is configured to be snugly or tightly received within the air inlet member of the intra-oral appliance.

13. The apparatus according to claim 11, wherein the adapter further comprises an internal passage support for holding the air passage in the outlet portion which is received in the air inlet member.

14. The apparatus according to claim 11, wherein the air outlet portion comprises an engagement formation releasably engaging the air inlet member of the intra-oral appliance, and the engaging formation is positioned at a terminal end of the air outlet portion, and extends behind an inner end of the air inlet member.

15. The apparatus according to claim 14, wherein the air inlet member is formed of a resilient and deformable material, and the air inlet member has a substantially constant elliptical cross-sectional configuration along its length that is configured to enable the patient's lips to form a seal about the air inlet member.

16. The apparatus according to claim 11, wherein the adapter comprises an adapter wall extending transversely outward beyond a periphery of the air inlet and air outlet portions, and the adapter wall is axially positioned between the air inlet and air outlet portions.

17. The apparatus according to claim 16, wherein the adapter wall has an elliptical configuration for corresponding to the shape of the mouth of a patient, and the adapter wall has a curvature across its surface that complements a curvature on the surface of the patient's face around their mouth.

18. The apparatus according to claim 16, wherein an outer wall of the appliance body and the adapter wall are configured to be positioned on respectively inner and outer sides of the patient's lips in use and help to resist the leakage of air out through a mouth opening of the patient.

19. The apparatus according to claim 11, wherein the air outlet member is configured to abut or be close to the patient's hard palate, and the air outlet member is formed of a resilient material that can be deformed in use into contact with the patient's hard palate.

20. A method of treating a patient with CPAP comprising fitting the intra-oral appliance and connected adapter as defined in claim 11 to a patient, the method comprising:
coupling the adapter to a CPAP machine; and
delivering positive air pressure from the CPAP machine to the patient through the adapter and intra-oral appliance into the upper airways of the patient to resist the obstruction of the upper airways of the patient.
